(19) United States
(12) Patent Application Publication
(10) Pub. No.: US 2015/0056310 A1
(43) Pub. Date: Feb. 26, 2015

(54) COSMETIC METHOD FOR INCREASING COLLAGEN EXPRESSION IN SKIN COMPRISING TOPICALLY APPLYING AN EXTRACT OF QUASSIA AMARA

(71) Applicant: BASF Beauty Care Solutions France S.A.S., Lyon (FR)

(72) Inventors: Valérie Cenizo, Lyon (FR); Valérie Andre, Ampuis (FR)

(21) Appl. No.: 14/463,882

(22) Filed: Aug. 20, 2014

(30) Foreign Application Priority Data
Aug. 23, 2013 (FR) 1358164

(51) Int. Cl. A61K 8/97; A61Q 19/08

(52) U.S. Cl. CPC A61K 8/97; A61Q 19/08
USPC 424/725

(57) ABSTRACT

The present invention relates to a cosmetic method for increasing collagen gene and/or protein expression comprising topically applying an effective amount of an extract of the plant Quassia amara onto skin and/or mucous membranes. The present invention relates as well to a cosmetic method comprising topically applying an effective amount of such an extract for increasing the firmness of skin and/or mucous membranes of an individual in need thereof.

The present invention also relates to a method of cosmetic care comprising topically applying a composition comprising an effective amount of the extract of Q. amara onto skin and/or mucous membranes, for increasing the collagen gene and/or protein expression.

The present invention relates to the cosmetic and/or dermatological use of a Quassia amara plant extract for increasing collagen gene and/or protein expression in the skin and/or the mucous membranes and/or increasing the firmness of the skin and/or of the mucous membranes.

Collagen is a constituent protein of the extracellular matrix (ECM) present in a large amount in vertebrate tissues. It is a broad family comprising 29 different types.

Among the various collagen types, type I collagen is in particular found in numerous human tissues such as tendons, ligaments, cornea and skin, located more specifically in the dermis. Type I collagen is the major collagen of skin.

Fibrillar collagen is formed from procollagen fibres, subsequently assembled into a network, and then stabilized by crosslinking. It is the collagen which gives the tissues their mechanical strength and, consequently, participates in maintaining their firmness.

During ageing of the skin, the amount of collagen decreases overall, thereby leading to a loss of firmness. This phenomenon is all the more pronounced since the skin is, inter alia, subjected to UV radiation. The term photobiological ageing is used.

Several mechanisms are involved in the reduction of the amount of collagen during tissue ageing: enzymes called collagenases are responsible for the degradation of collagen proteins. In parallel, a non-enzymatic glycation mechanism responsible for the formation of glycated proteins called AGEs (Advanced Glycation End Products), in the extracellular matrix, contributes to a decrease in the functionality of said matrix, and therefore of the skin.

Collagen is therefore the subject of numerous studies and innovations in the cosmetics field for the development and the improvement of cosmetic active agents aimed at maintaining the firmness of the skin.

Thus, cosmetic compositions said to have an anti-age or anti-ageing effect are known to those skilled in the art. Some cosmetic active agents inhibit, for example, the activity of collagenases, responsible for collagen degradation. Other active agents inhibit the collagen glycation mechanism.

However, such ingredients make it possible only to prevent collagen degradation and to maintain the amount thereof in the skin, but they do not make it possible to increase it or to increase the firmness of the skin.

The aim of the present invention is to provide a novel cosmetic and/or dermatological ingredient which makes it possible to increase collagen gene and/or protein expression, and/or to increase the firmness of the skin and/or of the mucous membranes.

This cosmetic and/or dermatological ingredient must be topically acceptable, easy to formulate and/or to use in a cosmetic and/or dermatological composition, and able to be administered topically, on all or part of the skin of the human body and/or of human mucous membranes.

Finally, the aim of the invention is to provide a method of cosmetic care by topical application of the composition containing the cosmetic ingredient for increasing collagen gene and/or protein expression in the skin and/or the mucous membranes and/or for increasing the firmness of the skin and/or of the mucous membranes.

Surprisingly, the inventors have discovered that a Q. amara plant extract increases collagen gene and/or protein expression, preferentially collagen protein expression, and more preferentially type I collagen protein expression.

In addition to this property, the Q. amara extract has the advantage of being topically acceptable, easy to formulate and/or to use in a cosmetic and/or dermatological composition and able to be administered topically, on all or part of the skin of the human body and/or of human mucous membranes.

Quassia amara is a tropical shrub of the family Simaroubaceae, 2 to 5 metres high, originating from Guiana, which is today found mainly in Central and South America. It is commonly referred to as Surinam quassia or amargo, often confused with Jamaican quassia.

The bark and the roots are known to be used in cases of anaemia or anorexia, or else cases of gastric dysfunction.

Quassia amara (Q. amara) is used orally in Europe and in the United States as a medicinal herb and is known to stimulate internal organs such as the liver and gallbladder.

The plant has a considerable bitterness, greater than that of quinine, when making a powerful non-stringent tonic. It is recommended and used in the United States for stimulating saliva and gastric juices.

The wood of Q. amara is also known for its antimicrobial properties.

Cosmetic uses of Q. amara extracts were already known. Thus, patent U.S. Pat. No. 8,293,291 describes a method for reducing the appearance of facial expression wrinkles by means of a composition containing one or more plant active agents, among which a Q. amara extract, via a mechanical effect on the facial muscles.

The Q. amara extract is in fact described as an inhibitor of the metabolism of acetylcholine, which is a neurotransmitter responsible for calcium release in muscle fibres, leading to the contraction thereof. This inhibition prevents muscle contraction, resulting in a “botox-like” effect.

Moreover, the invention described in application WO 2007/048985 filed by the applicant describes the use of a Q. amara wood extract which stimulates the expression of lysyl oxidases (LOXs), which are proteins involved in extracellular matrix (ECM) stabilization.

However, none of the known uses of a Q. amara extract neither describe nor suggest its collagen gene and/or protein expression-increasing properties, and therefore predict the present invention.

Thus, a subject of the present invention is the cosmetic and/or dermatological use, advantageously topically, of a Q. amara extract for increasing collagen gene and/or protein expression, in particular in the skin and/or the mucous membranes, and/or for increasing the firmness of the skin and/or of the mucous membranes.

For the purposes of the present invention, the expression “cosmetic use and/or composition and/or method” is intended to mean a non-pharmaceutical use and/or composition and/or method, i.e. which is not intended for therapeutic use and is applied to a part of the body which is said to be healthy, in particular to an area of the skin and/or of the mucous membranes which is said to be healthy.

For the purposes of the present invention, the term “healthy skin” or “healthy mucous membrane” is intended to mean an area of skin or of mucous membrane to which the extract according to the invention is applied and which is said to be “non-pathological” by a dermatologist, i.e. showing no infection, scar, skin disease or condition such as candidiasis, impetigo, psoriasis, eczema, acne or dermatitis, or wounds or injuries.

For the purposes of the present invention, the term “collagen” is intended to mean the type I, III, IV, V, VI, VII, XII, XIII, XIV, XVI, XVII, XXIV and XXIX collagen proteins present in the skin and/or the mucous membranes. Advantageously, this involves the type I, III, IV, V and VII collagen proteins, preferentially the type I, type III and type V collagen proteins and more preferentially the type I collagen protein. Preferentially, the collagen is human collagen, in particular of human skin and/or human mucous membranes.

Advantageously according to the invention, the term “type III collagen” is intended to mean the collagen protein present in the skin, and preferentially in the extracellular matrix and/or at the level of the wall of skin blood vessels.

For the purposes of the present invention, the term “type I collagen protein” is intended to mean the collagen protein present in the skin, the cornea, the tendons, the ligaments and the bones, more preferentially in the skin.

The present invention thus relates to the cosmetic and/or dermatological use, advantageously topically, of a Q. amara extract, in particular as an active ingredient, for increasing collagen gene and/or protein expression, preferentially type I collagen gene and/or protein expression, and/or for increasing the firmness of the skin and/or the mucous membranes.

For the purposes of the present invention, the term “collagen expression” is intended to mean collagen gene expression, i.e. mRNA (messenger RNA) expression and/or collagen protein expression.

Preferentially, it is collagen protein expression.

For the purposes of the present invention, the expression “increasing collagen gene and/or protein expression” is intended to mean an increase in the level of collagen gene and/or protein expression of at least 20%, preferentially of at least 40% and even more preferentially of at least 50%, relative to the level of collagen gene and/or protein expression measured in the absence of the Q. amara extract according to the invention.

Preferentially, the increase in expression is an increase in collagen protein expression and more preferentially type I collagen protein expression.

In one preferential embodiment of the invention, the Q. amara plant extract according to the invention is therefore used for increasing type I collagen protein expression in the skin.

According to one advantageous embodiment of the invention, it is an increase relative to the level of collagen gene and/or protein expression measured in dermal fibroblast cells cultured in vitro in the absence of the Q. amara extract according to the invention.

In one preferential embodiment of the invention, it is an increase in the level of type I collagen protein expression of at least 50% relative to the level of protein expression measured in dermal fibroblast cells cultured in vitro in the absence of the extract, according to the protocol as described, for example, in Example 2.

Advantageously, the measurement of the increase in protein expression is carried out by in vitro measurement, preferably by confocal microscopy measurement after immunolabelling for example according to the method as presented in Example 2.

For the purposes of the present invention, the term “immunolabelling” is intended to mean the technique consisting in binding an antibody specific for a given collagen protein, preferentially the type I collagen protein, to said protein, with a view to visualization by microscopic observation.

Advantageously, the visualization is carried out by confocal microscopy.

For the purposes of the present invention, the expression “increasing the firmness of the skin and/or of the mucous membranes” is intended to mean, from a cosmetic point of view, an increase, for aesthetic purposes, in the firmness of the skin and/or of the mucous membranes which have lost firmness, in particular under the effect of intrinsic factors such as tissue ageing of the skin and/or of the mucous membranes, i.e. chronological ageing, which is found, for example, in “mature” skin, i.e. the skin of individuals over the age of 40, cellular stress, non-pathological physiological variations, such as a change in diet, hormonal variations, in particular during puberty, pregnancy, menopause and andropause. This loss of firmness can also occur under the effect of extrinsic factors such as aggressive environmental agents, for instance UV radiation, pollution, smoke, tobacco, toxins, climatic aggressions and/or mechanical aggressions. It may in particular involve the cosmetic care and/or treatment of the unattractive appearance and/or uncomfortable aspect of stretch marks.

For the purposes of the present invention, the expression “increasing the firmness of the skin and/or of the mucous membranes” is intended to mean, from a dermatological point of view, an increase, for therapeutic purposes, of the firmness of the skin and/or of the mucous membranes which have lost firmness under the effect of pathological conditions of the skin and/or of the mucous membranes, for instance rosacea or telangiectasia.

The increase in firmness can be measured according to conventional methods, in particular by in vivo measurement via the fringe projection method using a cutometer, a densiscore, a torque meter or else a dynaskin combined with a dermatop.

According to the invention, the term “mucous membrane(s)” denotes the ocular mucous membrane, the vaginal mucous membrane, the urogenital mucous membrane and/or the buccal, in particular labial buccal, mucous membrane and/or the gingival mucous membrane, preferentially the ocular and/or buccal mucous membranes, and more preferentially the labial and/or ocular mucous membrane.

The Q. amara extract according to the invention is a topically acceptable cosmetic and/or dermatological extract.

For the purposes of the present invention, the expression “topically acceptable cosmetic and/or dermatological” is intended to mean an ingredient suitable for topical application, which is non-toxic, non-irritant to the skin and/or the mucous membranes, which does not induce an allergic response, and which is not chemically unstable.

For the purposes of the present invention, the term “topical application” of an ingredient is intended to mean the direct local application and/or the spraying of the ingredient on to the surface of the skin and/or of the mucous membranes.

The Q. amara extract according to the invention may be any extract of the plant chosen from the whole plant, the wood, the roots, the rhizome, the bark, the flowers, the petals, the sepals, the seeds, the fruits, the fruit skin, the areal parts, in particular the stem, the leaves, and mixtures thereof.

Advantageously, the Q. amara extract according to the invention is obtained by extraction of the Q. amara wood, more preferentially of the wood without bark. Advantageously, the extract according to the invention is extracted from the wood only, without bark.

The part of the plant is preferentially dried and/or ground before extraction.

The Q. amara extract according to the invention can be obtained by means of the plant extraction methods known in the field, for example by maceration of at least one part of the plant, preferably between 1% and 10% (w/w) of solids in a solvent or a mixture of solvents, preferably a protic polar solvent, and advantageously in water, an alcohol, a glycol, a polyol, or a water/alcohol, water/glycol or water/polyol mixture (such as water as a mixture with ethanol, glycerol, butylene glycol or other glycols, such as xylitol, etc.) of 100/0 to 0/100 (v/v).

The extracts obtained are then preferably centrifuged and/or filtered and/or distilled in order to recover the active soluble fraction (crude extract). Additional steps of decolourisation and/or deodorization can be carried out on the extract at any stage of the extraction and according to techniques known by those skilled in the art.

According to one preferential embodiment, the extract is obtained by aqueous extraction, preferably in water alone.

The extraction can be carried out for a period ranging from 1 hour to 20 hours, preferentially for a period ranging from 2 hours to 16 hours. More preferentially, said extraction is carried out for a period of 2 hours.

The extraction can be carried out at a temperature ranging from 2 to 25° C. Preferentially, said extraction is carried out at ambient temperature, i.e. at 20° C.

The extraction can be carried out by maceration, or by grinding using a bead mill, mortar grinding, ultrasonic grinding or grinding using a mixer.

Preferentially, the extraction is carried out by maceration.

The Q. amara extract can be obtained by extraction of an amount of from 1% to 10% by weight of solids from at least one part of the plant relative to the total weight of the mixture consisting of the solvent and of the plant part (w/w). Preferentially, the extract is obtained by extraction of an amount of 5% by weight of solids from at least one part of the plant, relative to the total weight of the mixture consisting of the solvent, preferentially water, and of the plant (w/w).

In one preferential embodiment of the invention, the extract is an aqueous extract obtained by maceration for a period of 2 hours at ambient temperature, therefore at 20° C., of 5% by weight of wood solids relative to the total weight of the mixture consisting of water and of the wood, in particular according to the protocol described in Example 1a).

In another preferential embodiment of the invention, the Q. amara extract is an aqueous extract obtained by aqueous maceration for a period of 16 hours at 4° C., of 5% by weight of wood solids relative to the total weight of the mixture consisting of water and of the wood, in particular according to the protocol described in Example 1b).

In yet another preferential embodiment of the invention, the Q. amara extract is obtained by maceration, in a water/butylene glycol (75/25; v/v) mixture, of 5% by weight of wood solids relative to the total weight of the mixture consisting of the water/butylene glycol mixture and of the wood, for a period of 2 hours at ambient temperature, in particular according to the protocol described in Example 1c).

In one alternative embodiment of the invention, the extract is an aqueous extract of leaf obtained by grinding, at ambient temperature, of 5% by weight of leaf solids relative to the total weight of the mixture consisting of water and of the leaves, in particular according to the protocol described in Example 1d).

The extract obtained according to the invention is preferably centrifuged in order to recover the soluble fraction. Preferentially, the supernatant obtained is then filtered (cut off threshold of 0.45 μm) using filters known to those skilled in the art.

The extract according to the invention can then be concentrated by evaporation of the solvent or dried, for example by lyophilisation or by spray-drying.

In one particular embodiment of the invention, in particular for use thereof in dermatology, the Q. amara extract obtained according to the invention may be sterilised.

The Q. amara extract according to the invention is then preferentially dissolved in a solvent, in particular a polar solvent, in particular chosen from water, an alcohol, a polyol, a glycol, such as pentylene glycol and/or butylene glycol, or a mixture thereof; it is advantageously a mixture of water and glycol such as butylene glycol and/or pentylene glycol.

According to one advantageous embodiment, the extract is thus solubilised in an aqueous vehicle, preferentially water. The extract is then used in accordance with the present invention, optionally after filtration.

According to the invention, the Q. amara extract can be used alone or in a cosmetic and/or dermatological composition intended for topical application, in particular to the skin and/or the mucous membranes of the human body.

According to one preferential embodiment, the extract is applied to at least one area of skin and/or of mucous membrane chosen from the neck, the neckline, the stomach, the arms, the thighs, the hips, the buttocks, the waist and/or the face, and preferentially the area around the eyes, and the lips.

The present invention therefore relates to the use of a cosmetic and/or dermatological composition which contains a Q. amara extract according to the invention, preferentially an aqueous extract of Q. amara wood, for increasing collagen gene and/or protein expression, preferentially type I collagen gene and/or protein expression, and/or for increasing the firmness of the skin and/or of the mucous membranes.

In the case of dermatological applications, the Q. amara extract according to the invention and/or the dermatological composition according to the invention is preferentially used for the dermatological care and/or treatment of pathological conditions involving a pathological loss of collagen gene and/or protein expression, in particular of type I collagen gene and/or protein expression, and/or a pathological loss of firmness, such as in the case of pathological conditions of the skin and/or of the mucous membranes, such as rosacea or telangiectasia.

The cosmetic and/or dermatological composition according to the invention advantageously contains the extract according to the invention at a concentration of between 1×10⁻⁴% and 10% by weight relative to the total weight of the composition. More advantageously, the composition according to the invention contains the extract according to the invention at a concentration of between 1×10⁻³% and 3%, more preferentially between 1×10⁻²% and 1% by weight relative to the total weight of the composition.

Moreover, the cosmetic and/or dermatological composition contains at least one topically acceptable cosmetic and/or dermatological excipient.

Advantageously, said excipient(s) is (are) chosen from at least one of the groups consisting of preservatives, emollients, emulsifiers, surfactants, moisturisers, thickeners, texturing agents, film-forming agents, pigments, stabilisers, solubilising agents, dyes and fragrances.

Again advantageously, the excipient(s) is (are) chosen from the group consisting of amino acids and derivatives thereof, polyglycerols, esters, cellulose polymers and derivatives, lanolin derivatives, phospholipids, lactoferrins, lactoperoxidases, sucrose-based stabilisers, vitamin E and derivatives thereof, xanthan gums, natural and synthetic waxes, vegetable oils, triglycerides, unsaponifiable compounds, phytosterols, plant esters, silicones and derivatives thereof, protein hydrolysates, jojoba oil and derivatives thereof, liposoluble/water-soluble esters, betaines, aminoxides, plant extracts, sucrose esters, titanium dioxides, glycines, and parabens, and more preferably from the group consisting of steareth-2, steareth-21, glycol-15 stearyl ether, cetearyl alcohol, phenoxyethanol, methylparaben, ethylparaben, propylparaben, butylparaben, butylene glycol, caprylyl glycol, natural tocopherols, glycerin, sodium dihydroxycetyl phosphate, isopropyl hydroxycetyl ether, glycol stearate, triisononanoin, octyl cocoate, polyacrylamide, isoparaffin, laureth-7, a carbomer, propylene glycol, hexylene glycol, glycerol, bisabolol, a dimethicone, sodium hydroxide, PEG-30 dipolyhydroxystearate, capric/caprylic triglycerides, cetearyl octanoate, dibutyl adipate, grapeseed oil, jojoba oil, magnesium sulphate, EDTA, a cyclomethicone, xanthan gum, citric acid, sodium lauryl sulphate, mineral waxes and oils, isostearyl isostearate, propylene glycol dipelargonate, propylene glycol isostearate, PEG-8, beeswax, hydrogenated palm kernel oil glycerides, lanolin oil, sesame oil, cetyl lactate, lanolin alcohol, castor oil, titanium dioxide, lactose, sucrose, low-density polyethylene, and an isotonic saline solution.

The cosmetic and/or dermatological composition of the invention can be chosen from an aqueous or oily solution, a cream or an aqueous gel or an oily gel, in particular a shower gel, a shampoo; a milk; an emulsion, a microemulsion or a nanoemulsion, which is in particular oil-in-water or water-in-oil or multiple or silicone-based; a mask; a serum; a lotion, a liquid soap; a dermatological bar; an ointment; a foam; a patch; a preferably liquid, pasty or solid anhydrous product, for example in the form of makeup powders, of a rod or of a stick, in particular in the form of a lipstick.

Advantageously, the composition of the invention is a cream or a serum, intended to be applied to specific parts of the body, such as the neck, the neckline, the stomach, the arms, the thighs, the hips, the buttocks, the waist and/or the face, and preferentially the area around the eyes, and the lips.

In addition, the cosmetic and/or dermatological composition of the present invention may contain one or more cosmetic active ingredients, resulting in a supplementary effect and/or a synergistic effect with the Q. amara extract according to the invention.

They may, for example, be anti-wrinkle ingredients, agents for stimulating fibroblast activity or proliferation, or agents for stimulating extracellular matrix molecules.

They may, moreover, be anti-ageing active agents and/or tensioning agents for a synergistic effect with the Q. amara extract. Advantageously, they are active ingredients which increase collagen gene and/or protein expression or active ingredients which prevent the degradation of said collagen, such as retinol, vitamin C, a Davilla rugosa extract sold under the name Collguard by BASF Beauty Care Solutions, a Hibiscus abelmoschus seed extract sold under the name Linefactor, a peptide sold under the name Dermican by the applicant, or else the products sold under the names Matrixyl, Matrixyl 300 and Regestril by the company Sederma.

The tensioning agents which can be used in the invention can be chosen from synthetic polymers, such as polyurethane latexes or acrylic latexes, polymers of natural origin, in particular polyholosides in the form of starch or in the form of carrageenans, alginates, agars, gellans, cellulose-based polymers and pectins; soya plant proteins and protein hydrolysates; mixed silicates; wax microparticles; inorganic colloidal filler particles chosen, for example, from silica, and silica-alumina composites; and also mixtures thereof.

Finally, they may be cosmetic ingredients, for instance antimicrobial agents, free-radical scavengers, soothing agents, calmatives or relaxants, agents which act on the microcirculation to improve the radiance of the complexion, in particular of the face, healing agents or slimming agents. Advantageously, the cosmetic and/or dermatological composition of the present invention also contains one or more tensioning agents and/or one or more antimicrobial agents and/or one or more free-radical scavengers and/or one or more soothing agents and/or one or more slimming agents and/or one or more agents which are active on the microcirculation.

Among the antimicrobial agents which can be combined with the Q. amara extract in the present invention, mention may be made of 2,4,4′-trichloro-2′-hydroxy diphenyl ether (or triclosan), 3,4,4′-trichlorobanilide, phenoxyethanol, phenoxypropanol, phenoxyisopropanol, hexamidine isethionate, metronidazole and salts thereof, miconazole and salts thereof, itraconazole, terconazole, econazole, ketoconazole, saperconazole, fluconazole, clotrimazole, butoconazole, oxiconazole, sulphaconazole, sulconazole, terbinafine, undecylenic acid and salts thereof, benzoyl peroxide, 3-hydroxybenzoic acid, 4-hydroxybenzoic acid, phytic acid, N-acetyl-L-cysteine acid, lipoic acid, azelaic acid and salts thereof, arachidonic acid, resorcinol, octoxyglycerine, octanoylglycine, caprylyl glycol, 10-hydroxy-2-decanoic acid, farnesol, phytosphingosines, and mixtures thereof.

The free-radical scavengers may be vitamin C and derivatives thereof, including ascorbyl glucoside, phenols and polyphenols, in particular tannins, ellagic acid and tannic acid; epigallocatechin and natural extracts containing the same, in particular green tea extracts; anthocyans; phenol acids, stilbenes; active agents for trapping monocyclic or polycyclic aromatic compounds, tannins such as ellagic acid and indole derivatives and/or active agents for trapping heavy metals, such as EDTA, free-radical scavengers such as vitamin E and derivatives thereof such as tocopheryl acetate; bioflavonoids; coenzyme Q10 or ubiquinone.

As soothing agents which are part of the composition of the invention, use may be made of pentacyclic triterpenes, ursolic acid and salts thereof, oleanolic acid and salts thereof, betulinic acid and salts thereof, salicylic acid salts, and in particular zinc salicylate, bisabolol, allantoin, omega-3 unsaturated oils, cortisone, hydrocortisone, indomethacin and betamethasone, anti-inflammatory active agents, and in particular those described in application FR 2847267, in particular the Pueraria lobata root extract sold under the name Inhipase™ by the applicant, and Theobroma cacao extracts.

The active ingredients which act on the microcirculation, vasoprotectives or vasodilators, can be chosen from flavonoids, ruscogenins, nicotinates and essential oils.

The slimming active agents can in particular be chosen from agents which inhibit lipoprotein lipase, such as those described in patent US 2003/086949 (Coletica) and in particular an extract of Peruvian liana (Uncaria tomentosa); draining active agents, in particular hesperitin laurate (Flavagrum™), or quercitin caprylate (Flavenger™); agents which inhibit the phosphodiestarase enzyme, agents which activate adenylate cyclase, cAMP and/or active agents capable of trapping spermine and/or spermidine. Mention may be made of a Coleus forskohlii root extract, a Cecropia obtusa extract, a Uva lactuca extract, caffeine, forskolin, theophylline, theobromine, and/or their derivatives, a hydrolysed kappa-carraghenan product known as Slimexcess™ sold by the applicant, and/or mixtures thereof.

Finally, the invention relates to a cosmetic care method comprising the topical application of the Q. amara extract according to the invention or of a cosmetic composition according to the invention to at least one area of the skin of the body, in particular of the human body, and/or the mucous membrane, in particular of human mucous membranes, for increasing collagen gene and/or protein expression, in particular type I collagen gene and/or protein expression, and/or increasing the firmness of the skin and/or of the mucous membranes.

In one embodiment of the invention, the cosmetic care method comprises the topical application of the Q. amara extract according to the invention in particular in the form of a cosmetic composition according to the invention. Preferentially, the cosmetic care method consists of the topical application of the active ingredient containing an aqueous extract of 5% (by weight relative to the total weight of the ingredient) of Q. amara wood (w/w) according to Example 3.

In another embodiment of the invention, the cosmetic care method comprises the topical application of the cosmetic composition according to the invention, containing the extract according to the invention at a concentration of between 1×10⁻⁴% and 10% by weight relative to the total weight of the composition. Again advantageously, the composition according to the invention contains the extract according to the invention at a concentration of between 1×10⁻³% and 3%, more preferentially between 1×10⁻²% and 1% by weight relative to the total weight of the composition.

Preferably, the cosmetic care method consists of the topical application of the Q. amara extract according to the invention or of the cosmetic composition containing said extract, to the area of skin and/or mucous membranes chosen from the neck, the neckline, the stomach, the arms, the thighs, the hips, the buttocks, the waist and/or the face, and preferentially the area around the eyes and/or the lips.

Preferentially, said method according to the invention is used for increasing type I collagen protein expression.

The cosmetic care method is also used for increasing the firmness of the skin and/or the mucous membranes.

The invention will be understood more clearly on reading the description of the figures and of the examples which follow.

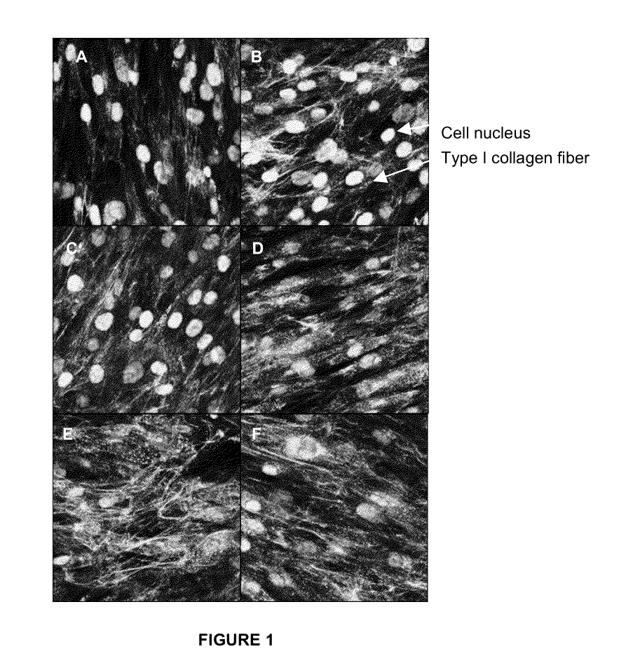

FIG. 1 represents the visualisation of the properties of the extract according to the invention on the increase in type I collagen protein expression by confocal microscopy during the experiment described in Example 2.

A. Nontreated control culture medium;

B. Culture medium treated with vitamin C (50 μg/ml);

C. Control culture medium treated with TGFβ1 (Transforming Growth Factor β1) (1 ng/ml);

D. Control culture medium treated with retinaldehyde (1 μm);

E. Culture medium treated with a Q. amara extract obtained according to Example 1a) at a final concentration of 0.01% (w/w);

F. Culture medium treated with a Q. amara extract obtained according to Example 1a) at a final concentration of 0.05% (w/w).

Examples referring to the description of the invention are presented hereinafter. These examples are given by way of illustration and could not in any way limit the scope of the invention. Each of the examples has a general scope. The examples are an integral part of the present invention and any feature appearing to be novel over any prior art on the basis of the description taken as a whole, including the examples, is an integral part of the invention.

### EXAMPLE 1

### Various Embodiments of the Invention for Obtaining a Q. amara Extract According to the Invention

a) Obtaining a Q. amara Wood Extract According to the Invention

Five percent of wood without bark, by weight of solids relative to the total weight of the mixture consisting of water and of the wood (w/w), of the Q. amara plant, were macerated in water, the water in this case being the only solvent, for a period of 2 hours at ambient temperature, i.e. at 20° C.

After maceration, the crude extract obtained was centrifuged for 10 min (8000 revolutions per min) and then the supernatant was recovered and filtered (cut-off threshold of 0.45 μm).

The wood extract at 5% by weight was then tested on the increase in type I collagen protein expression according to Example 2 hereinafter.

b) Obtaining a Q. amara Wood Extract According to the Invention

Five percent of wood without bark, by weight of solids relative to the total weight of the mixture consisting of water and of the wood (w/w), of the Q. amara plant, were macerated at a temperature of 4° C. for a period of 16 hours in water. The crude extract was then centrifuged for 10 min (8000 revolutions per min) and the supernatant was then filtered (0.45 μm).

c) Obtaining a Q. amara Wood Extract According to the Invention

Five percent of wood, by weight of solids relative to the total weight of the mixture consisting of water and of the wood (w/w), of the Q. amara plant, were macerated at ambient temperature, in this case 20° C., for a period of 2 hours, in a water/butylene glycol (75/25; v/v) mixture. The crude extract was then centrifuged for 10 min (8000 revolutions per min) and the supernatant was then filtered (0.45 μm).

d) Obtaining a Q. amara Leaf Extract According to the Invention

A leaf extract was obtained by grinding, in water, 5% of leaves, by weight of solids relative to the total weight of the mixture consisting of water and of the leaves (w/w). The grinding was carried out at ambient temperature, i.e. at 20° C. The ground extract was then centrifuged for 10 min (8000 revolutions per min) and the supernatant was then filtered (0.45 μm).

### EXAMPLE 2

### Demonstration of the Properties of the Extract According to the Invention on the Increase in Type I Collagen Protein Expression in Dermal Fibroblast Cells Cultured In Vitro

Material and Methods:

A Q. amara wood extract at 5% (w/w) obtained according to Example 1a) was tested on dermal fibroblast cells cultured in vitro. The extract was tested at final concentrations of 0.01% and 0.05% by volume in the culture medium. Preferentially, the final concentration was 0.01% (v/v).

Specifically, dermal fibroblasts from the abdomen of a 61-year-old donor were seeded into a culture chamber (8-well) and amplified in FGM (Fibroblast Growth Media) culture medium containing an antibiotic, normocin, for 3 or 4 days until confluence, the cell stage for which 100% of the cultured cells adhere to one another, forming a uniform cell layer.

The confluent cells were then treated in this same culture medium for 3 days with a Q. amara wood extract at a final concentration in the culture medium of 0.01% or 0.05% (v/v) (Table 1). Other control active ingredients were tested (Table 1): vitamin C (50 μg/ml) or retinaldehyde (1 μM) or TGFβ1 (1 ng/ml) (Transforming Growth Factor β1).

The culture media (FGM) containing the dermal fibroblast cells treated with an active agent, or the nontreated culture media, contained moreover 5 μg/ml of vitamin C, enabling the newly synthesized collagen to be secreted out of the cell.

After 3 days of incubation, the cells were rinsed with a phosphate buffered saline (PBS) buffer containing calcium, magnesium and antibiotics, and then fixed in cold methanol for 10 minutes with a view to performing immunolabelling.

An anti-type I collagen antibody was added to the medium with a view to performing immunolabelling.

The increase in type I collagen protein expression was observed by confocal microscopy (LSM 700, Zeiss, LePecq, France) and quantified by image analysis (FIG. 1).

The type I collagen protein expression was calculated as a ratio relative to a theoretical expression equal to 1 in the case of nontreated fibroblast cells (NT). The mean protein expression was obtained through the mean of 4 distinct assays (Table 1).

Results:

The results are given in Table 1 hereinafter and in FIG. 1.

TABLE 1
Measurement of the increase in type I collagen protein
expression in dermal fibroblast cells cultured in vitro
in the presence of various ingredients tested.

| Ingredients tested | Mean increase | Standard deviation |
|---|---|---|
| NonTreated (NT) | 1.00 | 0.75 |
| Retinaldehyde (1 μM) | 2.06 | 0.31 |
| Vitamin C (50 μg/ml) | 2.31 | 0.88 |
| TGFβ (1 ng/ml) | 1.42 | 0.93 |
| Q. amara (0.01%) | 2.35 | 0.48 |
| Q. amara (0.05% | 2.28 | 0.56 |

Discussions:

The results obtained with the usual positive controls for stimulation of type I collagen protein expression made it possible to validate the experiment.

Surprisingly, the aqueous extract of Q. amara wood at a final dose of 0.05% (v/v) made it possible to more than double the type I collagen protein expression (2.28 times), in comparison with the protein expression quantified in the nontreated cells.

When the concentration of the Q. amara wood extract was 0.01% (v/v), the increase in protein expression was 2.35 times that obtained in the nontreated cells.

### CONCLUSION

An increase in type I collagen protein expression was observed in the dermal fibroblast cells, cultured in vitro, treated with a Q. amara wood extract, particularly with a final dose of said extract in the culture medium of 0.01% (v/v).

### EXAMPLE 3

### Example of a Cosmetic and/or Dermatological Ingredient Containing the Q. amara Wood Extract According to the Invention

Methods known to those skilled in the art are carried out in order to mix together the various ingredients according to the present invention.

The Q. amara wood extract is the one obtained according to Example 1a). The % are percentages by weight relative to the total weight of the composition.

| | |
|---|---|
| Q. amara wood extract | 5% |
| Pentylene glycol | 5% |
| Butylene glycol | 20% |
| Keltrol (xanthan gum) | 0.5% |
| Water | qs 100 |

### EXAMPLE 4

### Example of a Cosmetic Composition Containing the Extract According to the Invention

The composition hereinafter is prepared according to methods known to those skilled in the art, in particular as regards the various phases to be mixed together.

| | |
|---|---|
| Cosmetic ingredient* | 3.00 |
| Xanthan gum | 0.50 |
| EDTA | 0.05 |
| Steareth-2 | 2.00 |
| Steareth-21 | 2.50 |
| Cetearyl alcohol | 1.00 |
| Propylheptyl caprylate | 15.00 |
| Sodium hydroxide (30% in solution) | 0.10 |
| Mixture of phenoxyethanol, | 1.25 |
| chlorphenesin, benzoic acid, | |
| butylene glycol, ascorbic acid | |
| (Germazide ™ PBS) | |
| Mixture of polyacrylate-X, | 4.00 |
| of isohexadecane and of | |
| polysorbate 60 (Sepigel ™ SMS 60) | |
| Water | qs 100 |

*The cosmetic ingredient is prepared according to Example 3 above. The amounts indicated are as percentage by weight relative to the total weight of the composition.

### EXAMPLE 5

### Example of a Dermatological Composition in Ointment Form Containing the Extract According to the Invention

The composition hereinafter is prepared according to methods known to those skilled in the art, in particular as regards the various phases to be mixed together. The amounts indicated are as percentage by weight relative to the total weight of the composition.

| | |
|---|---|
| Ingredient* | 3.00 |
| Excipient: Low-density polyethylene | 5.50 |
| Liquid paraffin | qs 100 |

*The ingredient is prepared according to Example 3 above. The ingredient according to the invention is sterilized and then dried before being incorporated into the composition in ointment form.

1. A cosmetic method for increasing collagen gene and/or protein expression comprising topically applying an effective amount of an extract of Quassia amara onto an area of the skin and/or mucous membranes of an individual in need thereof.

2. The cosmetic method according to claim 1, characterized in that the extract of Quassia amara is obtained by an aqueous extraction.

3. The cosmetic method according to claim 1, characterized in that the extract of Quassia amara is obtained from the wood.

4. The cosmetic method according to claim 3, characterized in that the extract of Quassia amara is obtained from the wood without bark.

5. The cosmetic method according to claim 1, for increasing the firmness of skin and/or mucous membranes.

6. The cosmetic method according to claim 1, characterized in that the collagen is type I collagen.

7. The cosmetic method according to claim 1, characterized in that the extract of Quassia amara is obtained by extraction of an amount of from 1 to 10% by weight of solids for at least one part of the plant relative to the total weight of the mixture solvent/plant.

8. The cosmetic method according to claim 7, characterized in that the extract of Quassia amara is obtained by extraction of an amount of 5% by weight of solids for at least one part of the plant relative to the total weight of the mixture solvent/plant.

9. The cosmetic method according to claim 1, characterized in that the area of the skin and/or mucous membranes is chosen in the group consisting of the neck, the neckline, the stomach, the arms, the thighs, the hips, the buttocks, the waist and/or the face.

10. The cosmetic method according to claim 9, characterized in that the area of the skin and/or mucous membranes is chosen from the area around the eyes and the lips.

11. The cosmetic method according to claim 1, characterized in that the extract of Quassia amara is dissolved in a polar solvent chosen in the group consisting of water, alcohol, polyol, glycol and any mixture thereof.

12. The cosmetic method according to claim 1, characterized in that the extract of Quassia amara is present in a cosmetic composition, said composition comprising a topically acceptable cosmetic excipient.

13. The cosmetic method according to claim 12, characterized in that the extract of Quassia amara is present in the composition at a concentration between 1·10⁻⁴% to 10% by weight relative to the total weight of the composition.

14. The cosmetic method according to claim 13, characterized in that the extract of Quassia amara is present in the composition at a concentration between 1·10⁻³% to 3% by weight relative to the total weight of the composition.

15. The cosmetic method according to claim 14, characterized in that the extract of Quassia amara is present in the composition at a concentration between 1·10⁻²% to 1% by weight relative to the total weight of the composition.

16. The cosmetic method according to claim 12, characterized in that the cosmetic composition also comprises one or more agents chosen in the group consisting of tensioning agents, antimicrobial agents, free-radical scavenging agents, soothing agents, slimming agents, agents active on microcirculation.

17. The cosmetic method according to claim 12, characterized in that the cosmetic composition is chosen in the group consisting of an aqueous solution, an oily solution, a cream, an aqueous gel, an oily gel, a milk, an emulsion, a microemulsion, a nanoemulsion, a mask, a serum, a lotion, a liquid soap, a dermatological bar, an ointment, a foam, a patch and an anhydrous product.
